(19) United States
(12) Patent Application Publication
(10) Pub. No.: US 2008/0095720 A1
(43) Pub. Date: Apr. 24, 2008

(54) Skin Benefit Compositions with a Vanilloid Receptor Antagonist

(75) Inventor: Helen Meldrum, Shelton, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/550,633

(22) Filed: Oct. 18, 2006

(51) Int. Cl. A61K 31/55; A61K 31/19; A61K 31/07; A61K 31/375; A61K 31/4415

(52) U.S. Cl. 424/59; 424/74; 424/744; 514/217.01; 514/557; 514/725; 514/474; 514/350

(57) ABSTRACT

Compositions that benefit skin and that reduce irritation of the skin are described. The compositions have a skin benefit agent like a hydroxy acid and a vanilloid receptor antagonist. The compositions of this invention can reduce wrinkles on the skin when applied, and they can be formulated to contain glycolic acid and capsazepine.

### FIELD OF THE INVENTION

The present invention is directed to a skin benefit composition comprising a vanilloid receptor antagonist. More particularly, the present invention is directed to a skin benefit composition comprising a vanilloid receptor antagonist and a skin benefit agent. The skin benefit composition of this invention may be applied topically to mammalian skin to yield a benefit, and unexpectedly, the composition does not produce a skin irritating or stinging effect generally associated with the skin benefit agent being used.

### BACKGROUND OF THE INVENTION

Many cosmetic compositions have been prepared to enhance the look of skin. Particularly, many consumers enjoy treating their skin in order to, for example, reduce wrinkles, eliminate age spots, reduce the look produced by freckles, lighten the skin, reduce the side effects of ultra-violet light, and the like. Unfortunately, however, many of the active ingredients in skin benefit compositions (i.e., the skin benefit agent) tend to irritate and in some cases even sensitize the skin, often making use of conventional skin benefit compositions undesirable. Moreover, attempts to make non-irritating compositions have been made, and however, conventional non-irritating compositions are typically not as effective as compositions that have skin benefit agents in an unadultered form or format.

It is of increasing interests to develop a skin benefit composition that is effective but that does not present any adverse side effects when being used. This invention, therefore, is directed to a skin benefit composition comprising a vanilloid receptor antagonist and a skin benefit agent. The skin benefit composition of this invention may be applied topically to mammalian skin to yield a benefit, and unexpectedly, the composition does not produce a skin irritating or stinging effect generally associated with the skin benefit agent being used.

Additional Information

Efforts have been disclosed for addressing vanilloid receptor ligands. In U.S. Patent Application No. 2006/030618 A1, vannilloid receptor 1 (VR1) antagonists are described.

Other efforts have been disclosed for addressing antagonists to the vanilloid receptor sub-type 1 (VR1) and uses thereof. In U.S. Patent Application No. 2006/0128710 A1, compounds particularly useful in the treatment of pain and inflammatory hyperalgesia are described.

Still other efforts have been disclosed for making cosmetic compositions. In WO 00/56270, cosmetic skin compositions with a weak carboxylic acid are disclosed.

None of the additional information above describes the use of a vanilloid receptor antagonist and a skin benefit agent in a skin benefit composition.

### SUMMARY OF THE INVENTION

In a first aspect, the present invention is directed to a skin benefit composition comprising:
(a) a skin benefit agent; and
(b) a vanilloid receptor antagonist.

In a second aspect, the present invention is directed to a method for topically treating skin with the skin benefit composition of the first aspect of this invention.

Irritating, as used herein, is meant to include inflammation, soreness or irritability of a body organ like skin (including stinging) such that a response or activity may be elicited. Irritating is meant to include sensory and/or visually detectable conditions, including conditions associated with redness, dryness and/or oedema. Stinging is meant to include a sharp and localized pain or essentially any negative sensation experienced by the consumer, including those that burn or itch.

Skin, as used herein, is meant to include skin on the face, neck, chest, back, arms, hands, legs and scalp. Skin benefit agent, as used herein, means an agent suitable to have a desired physical or biological effect on skin, whereby such effect includes moisturization and/or lightening of the skin and/or the reduction of wrinkles on the skin. Composition as used herein, is meant to include a composition that, preferably, is applied to the skin.

### DETAILED DESCRIPTION OF THE PREFERRED EMBODIMENTS

The only limitation with respect to the skin benefit agent that may be used in this invention is that the agent may be formulated in a composition comprising a vanilloid receptor antagonist. Illustrative and non-limiting examples of the types of skin benefit agents that may be used in this invention include retinoids, vitamin B₃, humectants, aloe vera, pigmentary or sunscreen grade metal oxides, vitamin C compounds, resorcinols, anti-sebum ingredients, hydroxy acids, surfactants, mixtures thereof or the like.

Illustrative retinoids that may be used include all natural and/or synthetic analogs of vitamin A or retinol-like compounds that possess the biological activity of vitamin A in the skin as well as the geometric isomers and stereoisomers of these compounds. The retinoid employed can be a C₂-C₂₄ alkyl ester of retinol, (such as retinyl palmitate, retinyl acetate, retinyl proprionate), retinal, retinaldehyde and/or retinoic acid. Such compounds are known and may be purchased from suppliers like Sigma Chemical Company, and Boerhinger Mannheim. When used, they typically make up from about 0.003 to about 5%, and preferably, from about 0.01 to about 3.5%, and most preferably, from about 0.01 to about 2.5% by weight of the composition, including all ranges subsumed therein.

The vitamin B₃ compound which may be used in this invention, if desired, includes water-soluble vitamin B₃ and any derivatives thereof that may be suitable for use within a skin benefit composition. Preferred vitamin B₃ compounds include niacinamide, nicotinic acid, nicotinyl alcohol, derivatives thereof, salts of any of the foregoing, as well as mixtures thereof. Typical derivatives of the vitamin B₃ compounds include nicotinyl amino acids, nicotinyl alcohol esters of carboxycilic acids, nicotinic acid N-oxide, niacinamide N-oxide and mixtures thereof. Preferably, however, when a vitamin B₃ compound is used, the compound is niacinamide. When used as a skin benefit agent, such a compound typically makes up from about 0.01 to about 15, and preferably, from about 0.02 to about 12, and most preferably, from about 0.03 to about 10% by weight of the skin benefit composition, including all ranges subsumed therein.

The humectant suitable for use in this invention is limited only to the extent that it may be used in a skin benefit composition. The humectants of the polyhydric alcohol type are often preferred. The humectant often aids by reducing scaling, stimulating the removal of buildup scale, and improving skin feel. Typical polyhydric alcohols that may be used in this invention include glycerol, polyalkylene glycols, and more preferably, alkylene polyols and their derivatives, including propylene glycol, dipropylene glycol, polypropylene glycol, polyethylene glycol and derivatives thereof, sorbitol, hydroxypropyl sorbitol, hexylene glycol, 1-3-butylene glycol, 1,2,6-hexanetriol, ethoxylated glycerol, propoxylated glycerol, mixtures thereof or the like. Other suitable humectants include glycerol quats, honey quats, sugar quats or mixtures thereof. Often, for best results the humectant is preferably propylene glycol. When employed, the amount of humectant typically used is often from about 1 to about 45, and preferably, from about 3 to about 30, and most preferably, from about 5 to about 18%, including all ranges subsumed therein.

Aloe vera (aloe barbadenis) in any form suitable for use in a skin benefit composition may be used as a skin benefit agent in this invention. Typically, the preferred form of aloe vera used herein is aloe vera gel, present in the skin benefit composition at a weight percent from about 0.0 to about 20, and preferably, from about 0.01 to about 15, and most preferably, from about 0.1 to about 12% by weight of the skin benefit composition, including all ranges subsumed therein.

The pigmentary or sunscreen grade metal oxides (e.g., titanium dioxide and zinc oxide) that may be used in this invention include those cosmetic grade oxides made available from suppliers like Eckart and Engelhard. Other sunscreens suitable for use herein include PABA, Ecamsule (Mexoryl), cinnamate, and salicylate. Avobenzophenone (Parsol 1789), octyl methoxycinnamate, 2-hydroxy-4-methoxyl benzophenone, octyl methoxycinnamate, 2-ethylhexyl-p-methoxy benzophenone are other preferred commercially available sunscreens which may be used in this invention. Still other sunscreens which may be used include octocrylene, butylmethoxydibenzoyl methane, phenyl bezimidazole sulfonic acid, mixtures thereof and the like. The exact amount of sunscreen which may be used in the composition of this invention can vary depending upon the degree of protection desired from the sun's UV radiation. Usually, however, from about 0.001 to about 25, and preferably, from about 0.1 to about 20% by weight of the composition is made up of sunscreen, including all ranges subsumed therein.

Vitamin C compounds may be used in this invention as a skin benefit agent and they include water soluble ascorbic acid salts and esters thereof. Such compounds include magnesium ascorbyl phosphate as well as sodium salts of the esters of ascorbic acid, including those made available from Roche Vitamins. When desired, vitamin C compounds typically make up less than about 5% by weight of the total weight of the skin benefit composition.

Skin lightening agents that may be used in this invention as a skin benefit agent include resorcinol and its derivatives like 4-substituted resorcinol, and di-substituted resorcinols such as those described in U.S. Application No. 2004/09832. When used, the resorcinol typically makes up from about 0.1 to about 12, and preferably, from about 0.15 to about 10, and most preferably, from about 0.3 to about 5% by weight of the skin benefit composition, including all ranges subsumed therein.

Anti-sebum ingredients which may be used as a skin benefit agent include talc, silicas and powders like those with a core powder, a hydroxyapatite layered over the core and a zinc layer directly over the hydroxyapatite. Such a powder, if desired for use, can make up from about 1 to about 45, and preferably, from about 3 to about 40, and most preferably, from about 5 to about 20% by weight of the composition, including all ranges subsumed therein.

In an especially preferred embodiment, the skin benefit composition of this invention comprises (as a skin benefit agent) a hydroxy acid, especially those like alpha-hydroxy acids, beta-hydroxy acids (e.g., salicylic acid), hydroxy carboxylic acids, and mixtures thereof and any combinations of their stereoisomers.

Preferably, the hydroxy acid is one having the general structure:

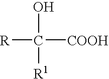

wherein R and R¹ are each independently hydrogen, or an unsaturated, straight or branched hydrocarbon chain comprising from about 1 to about 30 carbon atoms, or an aryl group.

The most preferred hydroxy acids suitable for use in this invention are lactic acid, 2-hydroxyoctanoic acid, hydroxylauric acid, glycolic acid and mixtures thereof whereby when sterioisomers exist, the L-isomer is often preferred. In an especially preferred embodiment, glycolic acid is the preferred hydroxy acid used.

When hydroxy acid is employed, the skin benefit composition typically comprises from about 0.5 to about 25, and preferably, from about 1 to about 20, and most preferably, from about 2 to about 15% by weight hydroxy acid, based on total weight of the skin benefit composition and including all ranges subsumed therein. The pH range of the resulting composition often is from about 2.5 to about 10 when such an acid is employed.

Regarding the vanilloid receptor antagonist that may be used in this invention, such an antagonist is limited only to the extent that the same may be used in a composition suitable for use by humans and able to block the effect of agonists (like capsaicin) of the vanilloid receptor (TRP-V1, VR-1). Illustrative examples of the type of antagonist suitable for use in this invention include those described in U.S. Patent Application No. 2006/003618 A1, the disclosure of which is incorporated herein by reference.

Other suitable antagonists that may be used in this invention include those described in U.S. Patent Application No. 2006/0128710 A1, the disclosure of which is incorporated herein by reference. Illustrative non-limiting examples of the types of antagonists that may be used include 7-benzyl-N-(4-tert-butylphenyl)-5,6,7,8-tetrahydropyrido[3,4-d] pyrimidine, N-(4-tert-butyl phenyl)-5,6,7,8-tetrahydropyrido[3,4-d] pyrimidin-4-amine, (2E)-N-(3,4-dimethoxyphenyl)-3-[4-tert-butyl)phenyl]prop-2-enamide, (2E)-3-[4-(tert-butyl)phenyl]-N-(4-hydroxy-3-methoxyphenyl)prop-2-enamide, (2E)-3-[4-(tert-butyl)phenyl]-N-(2-5,6,7,8-tetrahydronapthyl)prop-2-enamide or mixtures thereof. In a most preferred embodiment, however, the preferred antagonist suitable for use in this invention is a capsaicin receptor antagonist, and especially, capsazepine or derivatives or mimics thereof, including any combination thereof. Such compounds are known and may be made available from suppliers like AMGEN, Inc. Typically, the amount of vanilloid receptor antagonist used in the skin benefit composition of this invention is from about 0.05 to about 25, and preferably, from about 0.1 to about 20, and most preferably, from about 0.5 to about 10% by weight of the composition, including all ranges subsumed therein.

Commercially acceptable and conventional vehicles may be used to prepare the skin benefit compositions of this invention, acting as diluants, dispersants or carriers for the mixture of physical scatterers of this invention in order to yield the desired (end use) composition. Therefore, the vehicle for the composition described herein may be aqueous-based, anhydrous or an emulsion whereby a water-in-oil or oil-in-water emulsion is generally preferred. If the use of water is desired, water typically makes up the balance of the composition, and preferably, makes up from about 5 to about 98%, and most preferably, from about 65 to about 90% by weight of the composition, including all ranges subsumed therein.

In addition to water, organic solvents may be optionally included to act or assist as carriers within the compositions of the present invention. Illustrative and non-limiting examples of the types of organic solvents suitable for use in the present invention include alkanols like methyl, ethyl and isopropyl alcohol, mixtures thereof or the like.

Other optional additives suitable for use include oils like isopropyl myristate, cetyl myristate, 2-octyldodecyl myristate, avocado oil, almond oil, olive oil, neopentylglycol dicaprate, lanolin, cod liver oil, sunflower oil, soybean oil, palm oil, cottonseed oil, menhaden oil, coconut oil, castor oil, safflower oil, peanut oil, pine oil, rice bran oil, mixtures thereof or the like. Typically, such oils assist in emulsifying the skin benefit composition of this invention, and an effective amount is often used to yield a stable, and most preferably, water-in-oil emulsion.

Emollients may also be used, if desired, in the composition of the present invention. Alcohols like 1-hexadecanol (i.e., cetyl alcohol), behenyl alcohol and phenoxyethanol are often desired as are the emollients generally classified as silicone oils and synthetic esters Silicone oils suitable for use include cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material in the inventive topical composition described herein include polyalkyl siloxanes, polyalkylaryl siloxanes and polyether siloxane copolymers. The essentially non-volatile polyalkyl siloxanes useful herein include, for example, polydimethylsiloxanes with viscosities of from about 5 to about 25 million centistokes at 25° C. Among the preferred non-volatile emollients useful in the present compositions are the polydimethylsiloxanes having viscosities from about 10 to about 400 centistokes at 25° C.

The ester emollients that may optionally be used are:
(1) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) ether-esters such as fatty acid esters of ethoxylated fatty alcohols.
(3) polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) wax esters such as beeswax, spermaceti, stearyl stearate and arachidyl behenate.
(5) sterols esters, of which cholesterol fatty acid esters are examples.

While use of emollients is optional, the same can make up from about 0.1 to about 50% by weight of the composition, including all ranges subsumed therein. Fatty acids having from about 10 to about 30 carbon atoms may also be included in the compositions of this invention. Illustrative of this category are pelargonic, lauric, myristic, palmitic, stearic, isostearic, hydroxystearic, oleic, linoleic, ricinoleic, arachidic, behenic, erucic acids, and mixtures thereof.

Thickeners may also be utilized as part of the cosmetically acceptable carrier in the skin benefit composition of the present invention and they are often generally classified as carboxylic acid polymers, cross-linked polyacrylate polymers, polyacrylamide polymers or the like. Typical thickeners include cross linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose, polyacrylamide comprising thickeners (like Sepigel™305) and hydroxymethyl cellulose. Gums suitable for the present invention include guar, xanthan, magnesium aluminum silicate (Veegum), sclerotium, carrageenan, pectin and combinations of these gums. Amounts of the thickener may range from 0.0001 to 5%, usually from 0.001 to 1%, optimally from 0.01 to 0.5% by weight, based on total weight of the skin benefit composition and including all ranges subsumed therein.

Collectively, water, solvents, silicones, esters, fatty acids, humectants and/or thickeners often constitute the cosmetically acceptable carrier in amounts from about 1 to about 97.0%, preferably from 80 to 96.5% by weight, based on total weight of the skin benefit composition and including all ranges subsumed therein.

If desired, surfactants may be used in this invention as a skin benefit agent and they typically have an HLB of greater than about 8. Such surfactants are preferably hydrophilic and can include cationic, zwitheronic, anionic, non-ionic or amphoteric surfactants, including mixtures thereof. Illustrative surfactants are described in McCutcheon's Detergents and Emulsifiers, North American Edition (1986), published by Allured Publishing Corporation, the disclosure of which is incorporated herein by reference. Surfactants like glycerol monostearate are often preferred.

Many cosmetic compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives for use in this invention are methyl paraben, propyl paraben, phenoxyethanol and benzyl alcohol. Preservatives will usually be employed in amounts ranging from about 0.1% to about 2% by weight of the skin benefit composition.

Still other optional ingredients that may be used with the skin benefit composition of this invention include chelators (like EDTA), anti-oxidants, exfoliants and dispersants (e.g., PEG-100 stearate).

The skin benefit composition of the present invention is typically intended for use primarily as a product for topical application to human skin, especially as an agent for controlling wrinkles and other aging attributes on the skin. Often, the composition, when formulated to be topical, has a melting point from about 30° C. to about 45° C., a pH from about 2 to about 10.5, and most preferably, from about 4 to about 8, including all ranges subsumed therein.

When making the skin benefit composition of the present invention, the desired ingredients are mixed, in no particular order, and usually at temperatures from about 70 to about 85° C. and under atmospheric pressure. A capsule or tablet (for oral consumption) may be made via conventional techniques to include a skin benefit agent and the vanilloid receptor antagonist as described herein. Preferably, however, the skin benefit composition of this invention is a cream, liquid, ointment, spray, foam, mouse or a solid composition (e.g., a soap bar) and applied to the skin topically, preferably with a hydroxy acid and for wrinkle reduction.

The packaging for the topical composition of this invention can be a bottle, tube, roll-ball applicator, propellant driven aerosol device, squeeze container or lidded jar.

The examples below are provided to illustrate the invention and are not intended to limit the scope of the claims.

### EXAMPLE 1

A variety of over-the-counter skin creams generally believed to reduce wrinkles were assessed. Known brands sold under the L'Oreal®, Neutrogena® and Nivea® names were purchased and all purchased products at least contained a hydroxy acid and/or Vitamin A or one of its derivatives as a skin benefit agent.

Participants were asked to topically apply and use one product at a time in a manner consistent with the instructions provided with the respective product being used. Approximately 85% of the participants involved reported some form of noticeable irritation after using each product for more than three consecutive days.

### EXAMPLE 2

Keratinocyte cell lines (HaCaT) were treated with about 1 μM capsaicin (an agonist of vanilloid receptor TRP-V1, VR-1). Subsequent to treatment, a significant increase (p<0.001) in production of interleukin-1α was observed. Treatment of keratinocytes with capsaicin (1 μM) in the presence of about 3 μM capsazepine blocked the interleukin-1α increase in response to capsaicin, whereby capsazepine alone had no effect on interleukin-1α levels. The results demonstrate that agonists of TRP-V1 increase the release of pro-inflammatory mediators from keratinocytes and antagonists of TRP-V1 reduced the inflammatory cytokines. Antagonists of TRP-V1 are thereby expected to be a route to reduce inflammation in the skin when the same is subjected to an irritant.

### EXAMPLE 3

Topical skin formulations may be made with conventional carriers to comprise hydroxy acid (e.g., glycolic acid), vitamin A or a derivative thereof (e.g., retinal) and/or a resorcinol, and a vanilloid receptor antagonist (e.g., capsazepine). The formulations may be made with ingredients added in a manner consistent with the present discloser. Resulting formulations, which may be topically applied to participants, are not expected to result in noticeable irritation as reported by the participants in Example 1.

What is claimed is:

1. A skin benefit composition comprising:
(a) a skin benefit agent; and
(b) a vanilloid receptor antagonist.

2. The skin benefit composition according to claim 1 wherein the skin benefit agent is Vitamin A or a derivative thereof, Vitamin B₃, humectant, aloe vera, pigmentary or sunscreen grade metal oxide, Vitamin C or a derivative thereof, resorcinol, an anti-sebum ingredient, a hydroxy acid, or a mixture thereof.

3. The skin benefit composition according to claim 1 wherein the skin benefit agent is an alpha hydroxy acid, beta-hydroxy acid, hydroxy carboxylic acid, or a mixture thereof.

4. The skin benefit composition according to claim 1 wherein the skin benefit agent is lactic acid, 2-hydroxyoctanoic acid, hydroxylauric acid, glycolic acid or a mixture thereof.

5. The skin benefit composition according to claim 4 wherein the skin benefit agent makes up form about 0.5 to about 25% by weight of the skin benefit composition.

6. The skin benefit composition according to claim 1 wherein the composition further comprises a carrier.

7. The skin benefit composition according to claim 6 wherein the composition is a topical composition.

8. The skin benefit composition according to claim 1 wherein the vanilloid receptor antagonist is a capsaicin receptor antagonist.

9. The skin benefit composition according to claim 8 wherein the capsaicin receptor antagonist is capsazepine.

10. The skin benefit composition according to claim 1 wherein the vanilloid receptor antagonist makes up from about 0.05 to about 25% by weight of the skin benefit composition.

11. A method for improving the characteristics of skin comprising the step ingesting or contacting the skin with a skin benefit composition, the skin benefit composition comprising:
(a) a skin benefit agent; and
(b) a vanilloid receptor antagonist.

12. The method according to claim 11 wherein the skin benefit agent is Vitamin A or a derivative thereof, Vitamin B₃, humectant, aloe vera, pigmentary or sunscreen grade metal oxide, Vitamin C or a derivative thereof, resorcinol, an anti-sebum ingredient, a hydroxy acid, or a mixture thereof.

13. The method according to claim 11 wherein the skin benefit agent is an alpha hydroxy acid, beta-hydroxy acid, hydroxy carboxylic acid, or a mixture thereof.

14. The method according to claim 11 wherein the skin benefit agent is lactic acid, 2-hydroxyoctanoic acid, hydroxylauric acid, glycolic acid or a mixture thereof.

15. The method according to claim 11 wherein the skin benefit agent makes up form about 0.5 to about 25% by weight of the skin benefit composition.

16. The method according to claim 11 wherein the vanilloid receptor antagonist is a capsaicin receptor antagonist.

17. The method according to claim 16 wherein the capsaicin receptor antagonist is capsazepine.

18. The method according to claim 11 wherein the vanilloid receptor antagonist makes up from about 0.05 to about 25% by weight of the skin benefit composition.
